(19) United States
(12) Patent Application Publication
(10) Pub. No.: US 2008/0161659 A1
(43) Pub. Date: Jul. 3, 2008

(54) Sensor System for Measuring, Transmitting, Processing and Representing Physiological Parameters

(75) Inventors: Robert Reichenberger, Wunsiedel (DE); Gerd Kunze, Zwonitz (DE)

Correspondence Address: BROWDY AND NEIMARK, P.L.L.C.;624 NINTH STREET, NW, SUITE 300, WASHINGTON, DC, 20001-5303, US

(73) Assignee: RAUMEDIC AG, HELMBRECHTS (DE)

(21) Appl. No.: 11/817,215

(22) PCT Filed: Jan. 21, 2006
(86) PCT No.: PCT/EP2006/000526

§ 371 Date: Aug. 27, 2007

(30) Foreign Application Priority Data
Feb. 25, 2005 (DE) 10 2005 008 627.6

(51) Int. Cl. A61B 5/00

(52) U.S. Cl. 600/301

(57) ABSTRACT

A sensor system serves for measuring, transmitting, processing and representing physiological parameters. The sensor system has least one sensor for measuring at least one physiological parameter. At least one data processing module is in a signal connection with the sensor via a signal cable. A display device serves to represent the sensor data. An expansion module serves to transmit telemetry data. The expansion module may be connected to the data processing module via an interface on the latter. Via a cable-free telemetry line, the expansion module is connected to the at least one sensor or at least one additional sensor for measuring at least one physiological parameter. There results a sensor system in which the patient's freedom of movement is enhanced due to the possibility of using telemetry sensors.

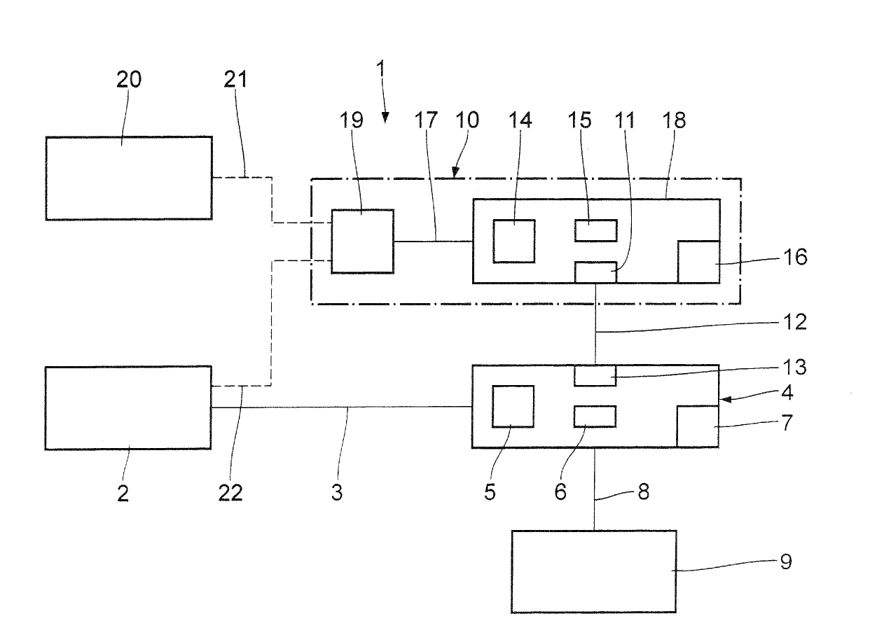

The invention relates to a sensor system for measuring, transmitting, processing and representing physiological parameters according to the preamble of claim 1.

This kind of sensor system is known from prior public use. During the application of this known sensor system, the patient is connected to corresponding sensors which are connected by cables to the data processing module. It is then possible to measure, transmit, process and represent the physiological parameters of the patient. A disadvantage is that the patient has to be stationary while being monitored during this procedure.

It is therefore an object of the present invention to develop a sensor system of the aforementioned kind such that its flexibility is enhanced and in particular the patient's freedom of movement is increased.

This problem is solved according to the invention by a sensor system with the features as outlined in the characterising part of claim 1.

According to the invention, it has been found that known basic sensor systems, which work with cable-connected sensors can be adapted for cooperation with non-cable-connected telemetry sensors if corresponding expansion components are provided. This makes use of the fact that, as a rule, the data processing module of the known cable-connected sensor systems has an interface for external data communication. In most cases this is a standard interface. The expansion module will then provide the possibility of expanding the known basic sensor system for communication with telemetry sensors already known per Se, for example, from WO 02/062 215 A2. Without necessitating in-depth changes to the data communication of the known sensor system, the data from telemetry sensors can now also be measured, transmitted, processed and displayed by the use of the additional expansion module. This makes it possible to make use of cable-free telemetry sensors for monitoring patients to supplement the use of cable-connected sensors. It is even possible to process the data from cable-free telemetry sensors exclusively without, in so doing, having to dispense with the proven components “data processing module” and “display device”. It is even conceivable, with the aid of the expansion module, simply to simulate the presence of an additional cable-connected sensor to the data processing module by means of a corresponding emulation. In this case, no intervention in the hardware or software of the known sensor system is even necessary. The sensor system is therefore able to be used very flexibly.

The expansion module according to claim 2 can take over work from the data processing module so that processing in the data processing module is accomplished more quickly and more efficiently. As an alternative, it is possible to create the expansion module purely as a transmission module without intermediate data processing, so that all the data processing takes place within the data processing module.

A subdivision of the expansion module according to claim 3 permits, for example, a read-out of the telemetry sensors directly on the patient's body with the help of the reader unit, so that the sensor radio modules of the telemetry lines can work with an advantageously low output. The reading in and transmission of the telemetry sensor data can then be done flexibly at different locations.

In the case of the expansion module according to claim 4, flexibility is again advantageously enhanced. For example, it is possible for the patient to carry a reader unit whilst going about his or her normal day-to-day routine. The reader unit can then relay the telemetry sensor data which have been read out to the transmission unit via the cable-free telemetry line. This can take place on the Internet, for example.

A bidirectional signal link according to claim 5 expands the possible applications of the sensor system considerably.

A control unit according to claim 6 permits sensor control or regulation through the data processing module or expansion module. The sensors, in other words the at least one cable-connected sensor and/or the at least one telemetry sensor, can then, for example, be initialized, modified in operation and/or adapted to cope with changes in the environment.

Sensors for measuring the physiological parameters listed in claim 7, can be used to advantage in the sensor system. This results in effective monitoring of patient parameters in different diagnostic and therapy situations.

At least one external sensor according to claim 8 is particularly suitable for calibrating the sensor system.

An arrangement of the external sensor according to claim 9 results in a compact sensor system.

A telemetry interface according to claim 10 has proven to be an advantageous standard for use in the sensor system.

An embodiment of the invention is explained in more detail below with reference to the drawing.

The sole FIGURE shows schematically a sensor system for measuring, transmitting, processing and representing physiological parameters.

The sensor system designated in entirety as 1 on the drawing comprises a sensor 2 for measuring at least one physiological parameter. Examples of such sensors are described in DE 102 39 743 A1 and in WO 02/062 215 A2. With the sensor 2, at least one of the following listed physiological parameters can be measured: pressure on the brain, oxygen content of body fluid or tissue, CO₂ content of body fluid or tissue, pH value of body fluid, body temperature, blood sugar content, blood flow. For measuring this at least one parameter, sensor 2 is connected to the head or body of the patient or implanted therein. A plurality of sensors 2 can also be provided for the sensor system 1.

Sensor 2 has a signal link via a signal cable 3 to a data processing module 4. The signal link via the signal cable 3 is bidirectional in design. The data processing module 4 serves to read in the sensor data and process said data, in other words serves particularly for recording, storing and analysing the sensor data. Additionally, the sensor 2 can be controlled or regulated with the help of the data processing module 4. The data processing module 4 has an integral control unit 5 for this purpose. For processing the data received by the sensor 2, the data processing module 4 has a processing unit 6, which has in particular a microprocessor and a memory.

In an embodiment which is not shown, the data processing module 4 is divided into a reader unit for reading in the sensor data and a data processing unit for reading out said data.

For detecting ambient parameters, at least one external sensor 7 is integrated into the data processing module 4. With the external sensor 7, the ambient air pressure and/or ambient temperature can be measured for calibrating the sensor system 1, for example.

The data processing module 4 is connected to a display and representation device 9, for example a PC, a laptop or a PDA, via a signal line 8. In an embodiment which is not shown, the display device 9 can also be integrated into the data processing module 4 or into the data processing unit thereof. The data relayed by the data processing module 4 is visualised and reworked as appropriate with the help of the display device 9.

With the at least one sensor 2, the data processing module 4 and the display device 9, the sensor system 1 is fully functional when using a cable-connected sensor 2. The sensor system 1 is expanded to integrate telemetry sensors for measuring at least one of the above-listed physiological parameters of the patient. In order to do this, the sensor system 1 has an expansion module 10, which is connected via an interface 11 and a signal line 12 to an interface 13 of the data processing module 4. The interfaces 11, 13 are standard interfaces, in particular RS232 interfaces. Corresponding to the respective components of the data processing module 4, the expansion module 10 also has a control unit 14, a processing unit 15 and an external sensor 16.

A transmission unit 18 of the expansion module 10 is connected to a reader unit 19 thereof via a signal line 17. The signal lines 8, 12, 17 may be signal cables or telemetry lines, in other words non-cable-connected signal links.

In the case of a cable-free signal link via the signal lines 8, 12, 17, the interface is designed as a Bluetooth interface.

The reader unit 19 is used for data communication of the expansion module 10 on the one hand with at least one telemetry sensor 20 via a cable-free first telemetry line 21, and on the other hand with the sensor 2 via a cable-free second telemetry line 22. The telemetry lines 21, 22 in particular may have a Bluetooth interface.

The transmission unit 18 serves to relay the sensor data received by the reader unit 19 to the data processing module 4. The relayed sensor data can already be pre-processed in the transmission unit 18 or fully prepared for representation in the display and representation device 9. In the latter case, the data processing module 4 serves purely as a transmission component for the sensor data from the transmission unit 18 to the display device 9. The processing unit 15 serves to process sensor data in the transmission unit 18.

It is also possible to control the sensor 2 or the telemetry sensor 20 via the expansion module 10. The expansion module 10 has the control unit 14 integrated into the transmission unit 18 for this purpose. The control data are transmitted via the signal line 17, the reader unit 19 and the corresponding telemetry line 22, 21 to the sensors 2, 20. Both the signal line 17 and the telemetry lines 21, 22 guarantee a bidirectional signal link.

For provision of the telemetry lines 21, 22, the sensors 2, 20 on the one hand and the reader unit 19 on the other hand have the necessary components which are known per se. The sensors 2, 20 are equipped with a trans-ponder which has, for example, a transmitting frequency of 13.56 MHz.

The reader unit 19 is equipped with corresponding components which work on this frequency, in other words a reader aerial and a receiver.

The sensor system 1 is used as follows: the basic system designed for measuring, transmitting, processing and representing physiological parameters acquired via cable-connected sensors 2, with the sensor 2, the data processing module 4 and the display device 9, is expanded with the help of the expansion module 10 and the at least one telemetry sensor 20 to form the sensor system 1. Depending on requirements, sensor data from cable-connected and/or cable-free linked sensors 2, 20 can now be measured, transmitted, processed and displayed using the sensor system 1. After expansion to form the sensor system 1, the basic system therefore comprises significantly expanded functions. Naturally it is possible to operate the sensor system 1 without using a single cable-connected sensor 2, thus enhancing the patient's freedom of movement.

1. A sensor system (1) for measuring, transmitting, processing and representing physiological parameters
with at least one sensor (2) for measuring at least one physiological parameter,
with at least one data processing module (4), which is in a signal connection with the sensor (2) via a signal cable (3), and
with at least one display device (9), which is in a signal connection with the data processing module (4), comprising an expansion module (10) for transmitting telemetry data which
can be connected to the data processing module (4) via an interface (13) provided thereon and
is in a signal connection via a cable-free telemetry line (21, 22) with the at least one sensor (2) or at least one additional sensor (20) for measuring at least one physiological parameter.

2. A sensor system according to claim 1, wherein the expansion module (10) has a processing unit (15) for processing the data received from the sensor (2, 20).

3. A sensor system according to claim 1, wherein the expansion module (10) has a reader unit (19) for data communication via the telemetry line (21, 22) and a transmission unit (18) separate therefrom and connected to the data processing module (4).

4. A sensor system according to claim 3, wherein the transmission unit (18) is connected to the reader unit (19) via a cable-free telemetry line.

5. A sensor system according to claims 1, wherein at least one of the data processing module (4) and the expansion module (19) is/are connected via a bidirectional signal link (3, 21, 22) to the at least one sensor (2) or to the at least one additional sensor (20).

6. A sensor system according to claims 1, wherein at least one of the data processing module (4) and the expansion module (10) has/have a control unit (5, 14) for controlling the sensor.

7. A sensor system according to claims 1, wherein the at least one sensor (2) or the at least one additional sensor (20) measures at least one of the following physiological parameters:
pressure on the brain,
oxygen content of body fluid or body tissue,
CO₂ content of body fluid or body tissue,
pH value of body fluid,
body temperature,
blood sugar content, and
blood flow.

8. A sensor system according to claims 1, comprising at least one further external sensor (7, 16) which measures at least one of the following ambient parameters:
air pressure, and
temperature.

9. A sensor system according to claim 8, wherein the external sensor (7, 16) is integrated into at least on of the data processing module (4) and/or the expansion module (10).

10. A sensor system according to claims 1, wherein the telemetry line (21, 22) has a Bluetooth interface.
